(19) United States
(12) Patent Application Publication
(10) Pub. No.: US 2006/0100616 A1
(43) Pub. Date: May 11, 2006

(54) Ultrasonic device

(75) Inventor: Jean M. Beaupre, Cincinnati, OH (US)

Correspondence Address: JAMES C. EAVES JR.;GREENEBAUM DOLL & MCDONALD PLLC, 3500 NATIONAL CITY TOWER, 101 SOUTH FIFTH STREET, LOUISVILLE, KY, 40202, US

(73) Assignee: Crescendo Technologies, LLC

(21) Appl. No.: 11/264,862

(22) Filed: Nov. 2, 2005

Related U.S. Application Data
(60) Provisional application No. 60/625,886, filed on Nov. 8, 2004.

(51) Int. Cl. A61B 18/04

(52) U.S. Cl. 606/34

(57) ABSTRACT

The present invention relates, generally, to ultrasonic medical devices and, more particularly, to ultrasonic surgical devices having improved cutting and cauterizing capabilities. In one embodiment, an ultrasonic waveguide (400) includes an amplifier that is convex and tapered in shape.

This application claims the benefit of Provisional Patent Application Ser. No. 60/625,886, filed on Nov. 8, 2004, to which priority is claimed pursuant to 35 U.S.C. §119(e) and which is hereby incorporated herein by reference.

### FIELD OF THE INVENTION

The present invention relates, generally, to ultrasonic medical devices and, more particularly, to ultrasonic surgical devices having improved cutting and cauterizing capabilities.

### BACKGROUND OF THE INVENTION

During surgery, a surgeon must both incise living tissue and control the resulting bleeding. This is traditionally done by cutting with a scalpel and tying off larger vessels with suture. This still leaves numerous smaller vessels to bleed. A very old technique of applying heat to wounds to stop bleeding is still used and is referred to as cauterization or coagulation.

A significant advance was the introduction of electrocautery instruments which pass a current through tissue to heat and cauterize the tissue as it is cut. The electric current itself may be used to cut tissue when properly controlled. However, electrocautery tends to desiccate and char tissue when applied at an intensity sufficient for cutting.

More recently, ultrasonic surgical devices have been introduced which permit effective cutting with reduced desiccation and charring. Initial work with these devices (Vang U.S. Pat. No. 2,714,890, Shafer U.S. Pat. No. 2,845,072, Balamuth U.S. Pat. No. 3,086,288) focused on improved cutting effects. In short, a vibrating cutting instrument would have advantages when incising tissue. Later work (Balamuth U.S. Pat. No. 3,636,943) noted that the heating action of a vibrating blade can also be used to control bleeding while cutting.

In current ultrasonic surgical devices, a power source, or generator, supplies a high frequency AC electrical signal to a hand held transducer. This transducer converts the electrical signal to longitudinal motion, as a standing wave, using piezoceramic, magnetostrictive, or similar means. The transducer may mechanically amplify this motion using a horn or horns for delivery to an end effector. The transducer and end effector are composed of an integer number of half-wave wave guides designed to vibrate in standing wave mode at the desired frequency. The end effector further amplifies the motion of the transducer, if necessary, to a useful level and transmits it to the functional portion of the device, which is shaped to perform a useful function. It is this end effector with its functional portion that, by action of its motion, cuts and cauterizes. Devices using this effect are available from Ethicon Endo-Surgery (Cincinnati, Ohio), for example.

With both cutting and cautery, the effect is proportional to the motion. As Balamuth noted and Vaitekunas, et al. confirmed, effects are linked to the velocity of the working end of the device. However, cutting and cautery effects can be considered as inverse to one another. If the device is very sharp, cutting will proceed very quickly and not allow as much heating of tissue, reducing cautery. As velocity increases, the force to cut is reduced and/or the cautery effect increases depending on the geometry of the device. Therefore, higher velocity is desirable.

Although providing an ultrasonic instrument with a velocity of greater than 17.44 m/s has been suggested, such as by Balamuth in U.S. Pat. No. 3,636,943, this disclosure does not account for the significant stress that accompanies the increased velocity. Although high velocities may be readily achieved by increasing the amplitude of a device at a given location, the functionality and life of these devices is severely limited by the debilitating strain placed on the instrument. In accordance with this, currently no ultrasonic device manufacturer claims to have a sustained velocity greater than 17.44 m/s, based on a published amplitude of 100 um at 55.5 kHz, with actual output maximum of 15.69 m/s, based on an amplitude of 90 um at the same frequency. Generally, ultrasonic devices have been limited to these velocities or less.

Currently, velocities above about 17.4 m/s are unavailable because the benefits of high velocity are outweighed by the increased stress placed on these instruments. Because velocity corresponds to the motion of the instrument, and an increase in motion is proportional to an increase in stress, the probability of blade failure generally increases as the velocity of the instrument is increased. Currently, the balance between stress and blade efficiency has resulted in instruments having velocities less than 17.44 m/s, based on a published amplitude of 100 μm at 55.5 kHz. It would therefore be advantageous to provide a high-velocity ultrasonic instrument having a stress level consistent with the safe application of the device.

### SUMMARY OF THE INVENTION

The present invention is directed to ultrasonic devices and, more particularly, to ultrasonic surgical devices having improved cutting and cauterizing capabilities. In one embodiment, an ultrasonic waveguide includes an amplifier that is convex and tapered in shape.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

The features of the invention may be set forth with particularity in the appended claims. The invention itself, however, both as to organization and methods of operation, together with further objects and advantages thereof, may best be understood by reference to the following description, taken in conjunction with the accompanying drawings in which:

FIG. 1 is a graph of stress versus cycles to failure in accordance with the present invention;

FIG. 2 is a graph of strain versus distance from the node in an instrument having a tapered horn;

FIG. 3 is a graph of strain versus distance from the node in an instrument having a stepped horn;

FIG. 4 is a graph of strain versus distance from the node in an instrument having a stepped horn, an instrument having a tapered horn, and an instrument having an approximate compound curve in accordance with the present invention;

FIG. 5 is a graph of strain versus distance from the node in an instrument having an approximate compound curve in accordance with the present invention; and

FIG. 6 is a side view of a distal half wave of an ultrasonic medical instrument.

### DETAILED DESCRIPTION OF THE INVENTION

Maximum motion, or velocity, can be calculated from V=πaf, where Velocity, V, is a function of Peak to Peak Amplitude, a, times the vibrating frequency, f, times π. Since most ultrasonic devices operate at a relatively fixed frequency, the amplitude must be increased to increase velocity. Most systems have a method of adjusting the amplitude by adjusting the output of the generator. This results, however, in a corresponding increase in the amount of stress the instrument is subjected to. Because, as a rule of thumb, a ten percent increase in stress corresponds to a decrease in useful life by an order of ten, high-velocity devices are generally impractical.

These limitations are due to the devices themselves being cyclically stretched, or strained, to their fatigue limits. The strain profile is sinusoidal or near sinusoidal in half waves. It is the cumulative axial strain over a distance in a single half wave between node and anti-node, or ¼ wave, that determines the amplitude.

$$a = 2 {\int }_{0}^{1 / 4 {λ}^{*}} ɛ ( x ) ⅆ x$$

The illustrated equation is provided with the node at x=0, ¼λ* the local ¼ wavelength, which may be different than that of a uniform bar, and ε(x) the local strain at a point x along the device.

Lower frequency devices generally operate with longer wavelengths, which allow them to accumulate more strain over the longer ¼ wavelength, and have a larger amplitude for a given local strain. However, this larger amplitude times the lower frequency gives the same velocity as a higher frequency device with its lower amplitude for the same strain. Therefore, velocity is constrained by maximum allowable strain and not frequency. This strain is largest at nodes, the middle of half-waves, and near zero at anti-nodes. As strain increases, the number of cycles necessary to cause a failure decreases logarithmically.

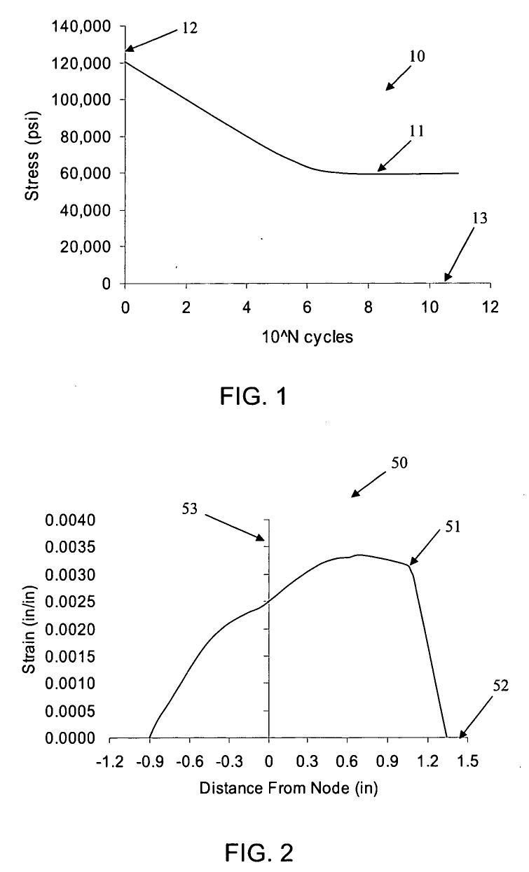

However, for some materials, such as titanium, if strain is kept below a particular threshold, the number of cycles before failure can be theoretically infinite. These limits are illustrated by S-N curves (FIG. 1) and are used to design products in fields ranging from aerospace to medical instruments. S-N relationships, as developed by Wohler, generally plot alternating stress (S) versus cycles to failure (N). The abscissa is generally stress and is plotted using a log scale and the ordinate is generally life to failure and is generally plotted using either a linear or log scale. Due to the high number of cycles an ultrasonic instrument may encounter, 10⁸ or more, the present invention includes ultrasonic devices having an optimal allowable strain over a ¼ wavelength.

For example, FIG. 1 illustrates one embodiment of an S-N graph 10 in accordance with the present invention. The ordinate 12 is stress, measured in psi and the abscissa 13 is logarithmic cycles to failure. The S-N curve 11 represents the endurance limit for a material given an applied level of alternating stress. Alternating stress levels below the S-N curve 11 will generally result in a low probability of material failure due to fatigue. Stress levels above the S-N curve 11 may overload the material resulting in low endurance and failure.

Increasing the velocity of ultrasonic instruments above the S-N curve generally can not be sustained and may result in a higher probability of blade failure. Because velocity is a function of strain, and the maximum strain is limited by the S-N curve, the present invention provides for maximizing the velocity of the instrument by maintaining a high level of strain throughout the length of a ¼ wavelength at an amplitude that corresponds to a low probability of instrument failure. This may be accomplished by designing an ultrasonic device with an elevated axial strain level, below a level that would cause premature failure, in the distal ¼ wave for a length sufficient to produce velocities exceeding 17.44 m/s.

By maintaining a level of strain at, for example 60,000 psi, for titanium, over the length of the distal ¼ wave, the present invention may increase the velocity of the instrument without increasing the strain on any one portion above the material's S-N curve. In accordance with the present invention, by maintaining a substantially consistent level of strain throughout a ¼ wavelength, as opposed to existing methods, which provide an elevated local increase in strain at one portion of the instrument, velocity may be substantially increased without a significant increase in stress. Therefore, the present invention maximizes the velocity of ultrasonic instruments, making cutting and/or cautery more efficient, while maintaining a stress level with a low probability of instrument failure.

There are 5 traditional types of horns, as defined by their profile, which are incorporated into ultrasonic instruments. Cross sections of these horns are generally square, rectangular, or circular due to ease of manufacture, but can be any shape. The 5 types are stepped, exponential, catenoidal, bessel, or conical; each according to its profile. Each horn may have a different effect on the physical properties of the ultrasonic instrument. For example, a stepped horn may be used as an amplifier that creates a rapid spike in amplitude. A conical horn may provide a more gradual increase in amplification across the length of the instrument.

Increasing the stress over the last ¼ wavelength of the ultrasonic instrument may be detrimental to the functionality and life of the instrument. The present invention includes using a compound horn, combining elements of traditional horns, to multiple horns in combination, over the last ¼ wave of the instrument. By maximizing the area under the curve ε(x) from 0 to ¼λ*, with ε(x) less than ε_{infinite life}, the velocity of the instrument is increased without the stress at any one portion of the instrument exceeding the S-N curve for the material. The material used in constructing ultrasonic devices in accordance with the present invention may be, for example, titanium and its alloys, aluminum and its alloys, stainless steel and its alloys, and ceramics. Each material will have a different S-N curve due to the characteristics particular to the use of that material. The present invention comprises determining the S-N curve for a material to be used in an ultrasonic instrument and using a compound horn to create a consistent strain at about the S-N curve or below to optimize the velocity of the instrument.

FIG. 2 illustrates a strain graph 50 demonstrating one example of the stress applied to an instrument having a tapered horn. The abscissa 52 may be the distance from the node in inches and the ordinate 53 may be the level of strain applied to the material. Strain curve 51, ε(x) represents one example of the varying material strain experienced across a ½ wavelength of an ultrasonic instrument incorporating a tapered horn. Although the material strain may reach levels, for example, of about 0.0032 in/in for titanium, the velocity of the instrument may not be optimized because the area under the curve ε(x) is not maximized. Providing an instrument with a conical horn exhibiting the strain curve 51 of FIG. 2 may not maximize the velocity over, for example, from about 0 to about 0.5 inches along the length of the material. Although the velocity of such instruments may be raised by increasing the peak of the strain curve, this increase in strain above the S-N curve may result in a higher probability of instrument failure.

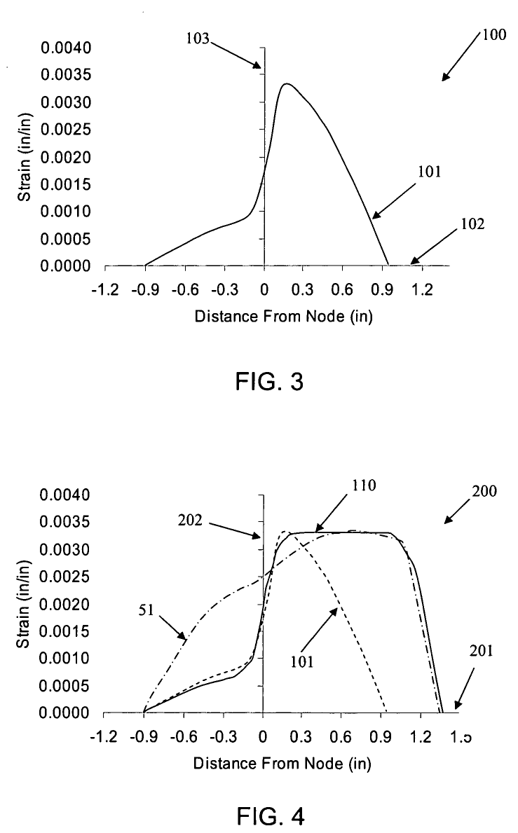

Similarly, radiused stepped horns are commonly used. A radiused stepped horn, depending on material properties, may display a strain curve 101, ε(x), as shown in FIG. 3. FIG. 3 illustrates one embodiment of a strain graph 100 for a stepped horn having an ordinate 103 that is strain (in/in) and an abscissa 102 that is distance from the node (inches). Strain curve 101 may represent the levels of strain across the length of the material generally attributable to the presence of a stepped horn. The strains of a pure stepped horn peak generally increase very rapidly at the step. Thus, a radius at the transition is often used to minimize the stress concentration. The peak strain of the ultrasonic instrument is generally sufficient for operation, however, an increase in velocity, with the use of a step alone, may require an increase in the strain of the device above the S-N curve for the material. Therefore, in current practice, velocity may be limited due to restrictions placed on the level of strain that may be applied to the material in order to maintain an acceptable instrument life. Consequently, high velocity levels may be unattainable in such devices because an elevated strain placed on a portion of the instrument will result in an undesirable high probability of blade failure.

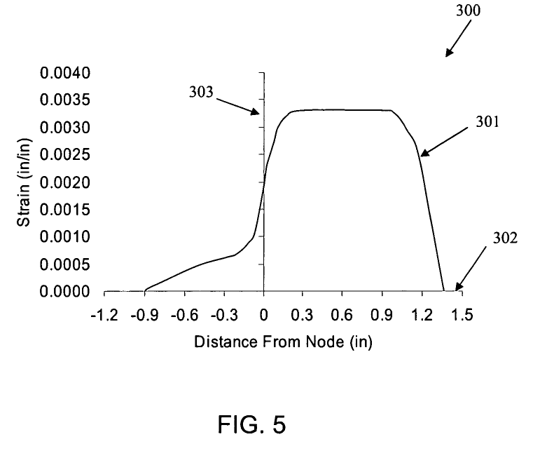

FIG. 4 illustrates strain curves for a stepped horn 51, a tapered horn 101, and for an approximate compound curve 110, ε(x) showing the level of strain provided by combining a conical horn with a radiused stepped horn. In accordance with the present invention, compound curve 110 combines the natural strain peaks of different ultrasonic horns such as, for example, a radiused stepped and conical horn, to maximize the area under the curve ε(x) from 0 to ¼λ*, with ε(x) less than ε_{infinite life}, such that the velocity of the instrument is increased without the stress at any one portion of the instrument exceeding the S-N curve for the material.. Rather than increasing the velocity of the instrument by increasing the peak strain of a single horn, the present invention maintains a substantially consistent level of strain, below the S-N curve, across a ¼ wavelength of the instrument illustrated in compound curve 301, ε(x), of FIG. 5. Providing consistent strain to increase velocity, rather than increasing the peak strain at a portion of the instrument, may increase the velocity of the instrument while maintaining a stress level within parameters that extend the useable life of the instrument.

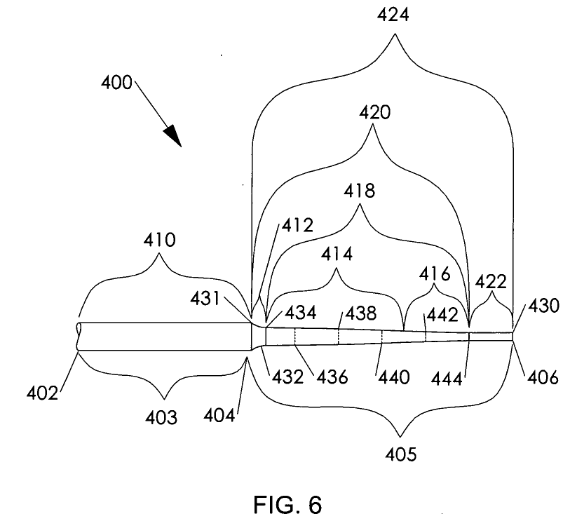

FIG. 6 illustrates one embodiment of a distal half wave 400 of an ultrasonic medical instrument. The distal half-wave 400 may include, in one embodiment, a proximal anti-node 402, where proximal anti-node 402 may be coupled to the waveguide (not shown) and the point at which the distal half-wave receives vibration. The distal half-wave 400 may include a shaft 410, where the shaft 410 may be proximal to, yet coupled with, the amplifier region 420 of the distal half-wave 400. The shaft 410 may transmit vibration from the connection point at proximal anti-node 402 to the amplifier region 420. The distal half-wave 400 may further include a functional portion 422, where the functional portion 422 may be distal to, yet coupled with, the amplifier region 420. Amplifier region 420 may provide high velocity vibratory motion that may be passed to the functional portion 422 for cutting and cauterization. The functional portion 422 may have any suitable configuration such as, for example, a ball configuration, a hook configuration, a paddle configuration, a curved configuration, a rod configuration, or a needle configuration. In a further embodiment of the present invention, the functional portion 422 may be a continuation of, for example, a tapered horn of the amplifier region 420.

Still referring to FIG. 6, distal half-wave 400 may include a proximal quarter wave 403 which may be defined by the region between proximal anti-node 402 and a node 404. Proximal quarter wave 403 may, for example, include only the shaft 410 or, in a further embodiment, portions of the amplifier region 420. The amplifier region 420 may, for example, begin at the node 404, at the distal end of the proximal quarter wave 403, and/or at the distal end of the shaft 410. The amplifier region 420 may take vibratory motion passing through the shaft 410 and amplify it to a suitable level for performing medical procedures. Amplified vibrations may then be passed to the functional portion 422 for cutting or cauterization. The amplifier region 420 may include a rapidly decreasing diameter portion 412. The rapidly decreasing diameter portion 412 may have, for example, a stepped radius configuration, an exponential configuration, a catenoidal configuration, or a distinct step configuration. Providing a rapidly decreasing diameter portion 412 may increase the strain on distal half wave 400, thereby increasing the velocity of the distal half wave 400. In one embodiment, the slope of the decrease in the diameter of the rapidly decreasing diameter portion 412 may be dimensioned to maintain a level of strain at about the S-N curve or below the S-N curve for the material used.

In one embodiment of the present invention, the amplifier region 420 may include a tapered portion 418 distal to, yet coupled with, the rapidly decreasing diameter portion 412. By providing, for example, a tapered portion 418 distal to the rapidly decreasing diameter portion 412, strain may be maintained at a substantially consistent level across the length of the distal half wave 400 by combining horns having different strain curves (FIGS. 2 and 3). In one embodiment of the present invention, the tapered portion may be dimensioned to maintain a level of strain at about the S-N curve or below the S-N curve for the material. Because the rapidly decreasing diameter portion 412 may display a rapidly peaking strain curve, such as the strain curve of FIG. 2, and the tapered portion 418 may display a more gradual sloping strain curve, such as the strain curve of FIG. 3, providing a compound horn may combine the differing strain curves to establish a more consistent hybrid strain curve. Combining the rapidly decreasing diameter portion 412 and the tapered portion 418 at dimensions below the S-N curve for the material may allow for a substantially consistent level of strain across the last quarter wave of the instrument that is at about the S-N curve or below the S-N curve for the material. Rather than providing a high level of acute peak strain, the present invention may provide a level of consistent strain, thereby producing a high velocity, while still maintaining a theoretically infinite life for the material at about or below the S-N curve. In a further embodiment, the present invention includes providing, for example, only a tapered portion tailored to provide a level of strain at about or below the S-N curve for the material. The tapered portion may be provided with, for example, a convex portion to maintain a suitable level of strain.

Tapered portion 418 may include a proximal portion 414 having, for example, a straight or convex profile. Stress variation along the proximal portion 414 may be uniform or substantially uniform. Proximal portion 414 may provide a great deal of cumulative strain, thereby increasing the amplitude of the functional portion 422. Tapered portion 418 may, for example, further include a distal portion 416 that may have, for example, a straight, convex, or concave profile. Tapered portion 418 may include any suitable configuration for providing a substantially consistent level of strain at about the S-N curve or below the S-N curve. Providing a tapered portion 418 with, for example, a convex portion, may facilitate providing a strain curve at about the S-N curve or below the S-N curve for the material. The distal quarter wave is herein defined as the region between node 404 and the anti-node 406 located at the distal end of the medical device.

Providing a compound horn such as, for example, a medical device combining a rapidly decreasing diameter portion 412 with a tapered portion 418 may combine dissimilar strain curves associated with different horns to maximize the level of strain across the instrument, rather than increasing the peak strain at any single location to achieve a high velocity. Distributing a high level of strain, at about the S-N curve or below the S-N curve, across the distal quarter wavelength 405 of the medical device may provide a high level of velocity while retaining a long useful life. Although specific examples will be detailed herein, it will be apparent to one of ordinary skill in the art that multiple horns, having various strain curve characteristics, may be combined into a compound horn in order to provide a level of strain substantially at about or below the S-N curve for any suitable material. The compound horns disclosed are described by way of example only and are not intended to limit the scope of the invention.

For example, still referring to FIG. 6, in one embodiment of the present invention, the length of the distal half wave 400 is 2.17 inches. The length of the shaft 410 is 0.87 inches with a diameter of 0.140 inches at the proximal end. The length of the rapidly decreasing diameter portion 412, the tapered portion 418, and the functional portion 422 is 1.30 inches. The length of the rapidly decreasing diameter portion 412 is 0.072 inches from the distal end of the shaft 410, with a radius of 0.125 inches. The diameter of the rapidly decreasing diameter portion 412 at the distal end is 0.09 inches and is 0.217 inches from the distal end of the shaft 410. The diameter of point 436, which is 0.217 inches from the distal end of shaft 410, is 0.087 inches. The diameter of point 438, which is 0.217 inches from point 436, is 0.079 inches. The diameter of point 440, which is 0.217 inches from point 438, is 0.065 inches. The diameter of point 442, which is 0.217 inches from point 440, is 0.050 inches. The diameter of point 444, which is 0.217 inches from point 442, is 0.040 inches. The length of functional portion 422 is 0.217 inches. The diameter of the distal portion 422 is 0.040 inches at the distal end.

In a further example of the present invention, the length of the shaft 410 is 0.87 inches with a diameter of 0.250 inches at the proximal end. The length of the rapidly decreasing diameter portion 412, the tapered portion 418 and the functional portion 422 is 1.40 inches. The length of the rapidly decreasing diameter portion 412 is 0.051 inches from the distal end of shaft 410, with a radius of 0.06 inches. The diameter of point 436, which is 0.200 inches from the distal end of shaft 410, is 0.114 inches. The diameter of point 438, which is 0.200 inches from point 436, is 0.100 inches. The diameter of point 440, which is 0.200 inches from point 438, is 0.080 inches. The diameter of point 442, which is 0.200 inches from point 440, is 0.056 inches. The diameter of point 444, which is 0.200 inches from point 442, is 0.040 inches. The length of functional portion 422 is 0.200 inches. The diameter of the distal portion 422 is 0.040 inches at the distal end.

In a further example of the present invention, the length of the shaft 410 is 0.55 inches with a diameter of 0.140 inches at the proximal end. The length of the rapidly decreasing diameter portion 412, the tapered portion 418 and the functional portion 422 is 1.45 inches. The length of the rapidly decreasing diameter portion 412 is 0.077 inches, from the distal end of the shaft 410, with a radius of 0.125 inches. The diameter of point 436, which is 0.242 inches from the distal end of shaft 410, is 0.083 inches. The diameter of point 438, which is 0.242 inches from point 436, is 0.075 inches. The diameter of point 440, which is 0.242 inches from point 438, is 0.064 inches. The diameter of point 442, which is 0.242 inches from point 440, is 0.050 inches. The diameter of point 444, which is 0.242 inches from point 442, is 0.040 inches. The length of functional portion 422 is 0.242 inches. The diameter of the distal portion 422 is 0.040 inches at the distal end.

While the invention has been described in connection with particular ultrasonic constructions, various other devices and methods of practicing the invention will occur to those skilled in the art. Accordingly, the scope of the present invention should not be limited by the particular embodiments described above, but should be defined only by the claims set forth below and equivalents thereof.

What is claimed is:

1. An ultrasonic waveguide, comprising:
an amplifier region, the amplifier region having a profile that is convex in shape and tapered in shape.

2. The ultrasonic waveguide of claim 1, wherein said ultrasonic waveguide comprises titanium.

3. The ultrasonic waveguide of claim 1, wherein said ultrasonic waveguide is smaller than about 10 millimeters in diameter.

4. The ultrasonic waveguide of claim 1, wherein said ultrasonic waveguide is configured to reciprocally vibrate in resonance at a frequency between about 20 and about 100 kHz.

5. The ultrasonic waveguide of claim 1, wherein stress of said ultrasonic waveguide does not exceed about 60 ksi.

6. The ultrasonic waveguide of claim 1, wherein stress of said ultrasonic waveguide does not exceed about 80 ksi.

7. The ultrasonic waveguide of claim 1, wherein said waveguide comprises an end-effector.

8. An ultrasonic waveguide, comprising:
an amplifier region, the amplifier region having a profile that is tapered in shape, the tapered shape having a convex shape.

9. The ultrasonic waveguide of claim 8 wherein said ultrasonic waveguide comprises titanium.

10. The ultrasonic waveguide of claim 8 wherein said ultrasonic waveguide is smaller than about 10 millimeters in diameter.

11. The ultrasonic waveguide of claim 8 wherein said ultrasonic waveguide operates at a frequency between about 20 and about 100 kHz.

12. The ultrasonic waveguide of claim 8 wherein stress of said ultrasonic waveguide does not exceed about 60 ksi.

13. The ultrasonic waveguide of claim 8 wherein stress of said ultrasonic waveguide does not exceed about 80 ksi.

14. The ultrasonic waveguide of claim 8 wherein said waveguide comprises an end-effector.

15. An ultrasonic waveguide, comprising:
an amplifier region, the amplifier region having a profile, and said profile having at least one portion that is convex in shape and tapered in shape.

16. The ultrasonic waveguide of claim 15, wherein said ultrasonic waveguide comprises titanium.

17. The ultrasonic waveguide of claim 15, wherein said ultrasonic waveguide is smaller than about 10 millimeters in diameter.

18. The ultrasonic waveguide of claim 15, wherein said ultrasonic waveguide is configured to reciprocally vibrate in resonance at a frequency between about 20 and about 100 kHz.

19. The ultrasonic waveguide of claim 15, wherein stress of said ultrasonic waveguide does not exceed about 80 ksi.

20. The ultrasonic waveguide of claim 15, wherein said waveguide comprises an end-effector.
